(19) United States
(12) Patent Application Publication
(10) Pub. No.: US 2005/0244460 A1
(43) Pub. Date: Nov. 3, 2005

(54) Biodegradable crosslinking strategies using triglycidyl amine (TGA)

(76) Inventors: Ivan Alferiev, Clementon, NJ (US); Robert J. Levy, Merion Station, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/119,063

(22) Filed: Apr. 29, 2005

Related U.S. Application Data
(60) Provisional application No. 60/566,565, filed on Apr. 29, 2004.

(51) Int. Cl. A61F 2/02; C08G 65/04

(52) U.S. Cl. 424/426; 623/23.72; 528/419

(57) ABSTRACT

The invention relates to implantable biodegradable bioprostheses and methods for making and using the bioprostheses. The implantable biodegradable bioprosthesis includes biomolecules having a reactive moiety and optionally a reactive group; and a biodegradable cross-linking moiety having (a) at least two linking moieties, wherein the at least two linking moieties are non-biodegradable and (b) a spacer, wherein the spacer is biodegradable and is in communication with the at least two linking moieties, provided that the biodegradable cross-linking moiety is artificial and is covalently bound to the reactive moiety. The implantable bioprosthesis is adapted to sufficiently degrade upon exposure to a cell or an enzyme to permit an expansion of the implantable bioprosthesis.

### BACKGROUND OF THE INVENTION

1. Field of Invention

The field of the invention is remodeling and stabilization of implantable bioprosthetic devices and tissues.

2. Description of Related Art

Surgical implantation of prostheses and tissues derived from biological sources, collectively referred to herein as bioprosthetic devices or bioprostheses, is an established practice in many fields of medicine. Common bioprosthetic devices include heart valves, pericardial grafts, cartilage grafts and implants, ligament and tendon prostheses, vascular grafts, skin grafts, dura mater grafts, and urinary bladder prostheses. In the case of valvular prosthetic devices, bioprostheses may be more blood compatible than non-biological prostheses and do not require anticoagulation therapy.

Bioprosthetic devices include prostheses, which are constructed entirely of animal tissue, and combinations of animal tissue and synthetic materials. Furthermore, a biological tissue used in a bioprosthetic device can be obtained or derived from the recipient (autogeneic), from an animal of the same species as the recipient (allogeneic), from an animal of a different species (xenogeneic), or alternatively, from artificially cultured tissues or cells.

Irrespective of the source of the tissue, major objectives in designing a bioprosthetic device include enhancement of durability and reduction of biomechanical deterioration in order to enhance the functional endurance of the device.

The material stability of bioprosthetic devices can be compromised by any of several processes in a recipient, including, for example, immune rejection of the tissue, mechanical stress, and calcification. Implantation of biological tissue that is not pretreated (i.e. stabilized prior to implantation) or is implanted without prior suppression of the recipient's immune system can induce an immune response in the recipient directed against the tissue. Identification of bioprosthetic tissue as “non-self” by the immune system can lead to destruction and failure of the implant. Even in the absence of an immune response, mechanical stresses on implanted tissue can induce changes in the structure of the bioprosthesis and loss of characteristics important to its mechanical function. In addition to these degradative processes, calcification of bioprosthetic tissue (i.e. deposition of calcium and other mineral salts in, on, or around the prosthesis) can substantially decrease resiliency and flexibility in the tissue, and can lead to biomechanical dysfunction or failure. In order to extend the useful life of bioprosthetic devices by improving their mechanical properties and mitigating their antigenic properties, the devices can be treated prior to implantation using a variety of agents. These pretreatment methods are collectively referred to in the art as fixation, cross-linking, and stabilization.

Glutaraldehyde is the most common stabilizing reagent used for treatment of valvular and other collagen-rich bioprosthetic devices. Glutaraldehyde is a cross-linking agent which has been used for pre-implantation stabilization of tissues, both alone and in combination with a variety of other reagents including diisocyanates, polyepoxide ethers, and carbodiimides. Pretreatment using glutaraldehyde and, optionally, other reagents, stabilizes implantable tissue with respect to both immune reactivity and mechanical stress by covalently linking proteins and other structures on and within the tissue. Cross-linking of a bioprosthetic tissue can be accompanied by treatment with an additional reagent (e.g. ethanol) to retard post-implantation calcification of the tissue. Use of glutaraldehyde as a stabilizing reagent can accelerate prosthesis calcification and necessitates use of a calcification inhibitor. Known calcification inhibitors include ethanol, aluminum salts such as, for example, aluminum chloride, chondroitin sulfate, and aminopropanehydroxyphosphonate (APD).

Even though durability has always been a concern in designing implants, in some cases relative growth of the implant would be desirable, e.g. a heart valve prosthesis implanted in a young child. In such cases, the ability to control enzymatic degradation of implants becomes an important tool. Therefore, despite the foregoing developments, there is a need for bioprosthetic devices capable of controlled enzymatic degradation resulting in cell in-growth remodeling of the bioprosthetic devices. The present invention provides such bioprosthetic devices and methods of making and using thereof.

All references cited herein are incorporated herein by reference in their entireties.

### BRIEF SUMMARY OF THE INVENTION

Accordingly, the invention provides an implantable bioprosthesis comprising biodegradably and artificially cross-linked biomolecules.

Also provided is an implantable bioprosthesis comprising biomolecules having a reactive moiety and optionally a reactive group, and a biodegradable cross-linking moiety having (a) at least two linking moieties, wherein the at least two linking moieties are non-biodegradable and (b) a spacer, wherein the spacer is biodegradable and is in communication with the at least two linking moieties, provided that the biodegradable cross-linking moiety is artificial and is covalently bound to the reactive moiety, whereby the biomolecules are cross-linked.

In certain embodiments, the implantable bioprosthesis is adapted to sufficiently degrade upon exposure to a cell or an enzyme at a biodegradation rate to permit an expansion of the implantable bioprosthesis and wherein the biodegradation rate is affected by an amount of the spacer.

In certain embodiments, the implantable bioprosthesis is a member selected from the group consisting of an artificial heart, a heart valve prosthesis, an annuloplasty ring, a dermal graft, a vascular graft, a vascular stent, a structural stent, a vascular shunt, a cardiovascular shunt, a dura mater graft, a cartilage graft, a cartilage implant, a pericardium graft, a ligament prosthesis, a tendon prosthesis, a urinary bladder prosthesis, a pledget, a suture, a permanently in-dwelling percutaneous device, a surgical patch, a vascular stent, a cardiovascular stent, a structural stent, a coated stent, a vascular shunt, a cardiovascular shunt, and a coated catheter. Preferably, the implantable bioprosthesis is the heart valve prosthesis. In certain embodiments, the biomolecules are derived from a biological tissue and or a synthetic analog of a bioprosthetic tissue. In certain embodiments, the tissue is selected from the group consisting of a heart, a heart valve, an aortic root, an aortic wall, an aortic leaflet, a pericardial tissue, a connective tissue, dura mater, a bypass graft, a tendon, a ligament, a dermal tissue, a blood vessel, an umbilical tissue, a bone tissue, a fascia, and a submucosal tissue. In certain embodiments, the tissue is harvested from an animal. Preferably, the animal is selected from the group consisting of a human, a cow, a pig, a dog, a seal, and a kangaroo. In certain embodiments, the biomolecules are members selected from the group consisting of proteins, glucosoaminoglucans, and an extracellular matrix.

Further provided is a method of making the implantable bioprosthesis of the invention, the method comprising providing an untreated bioprosthesis comprising biomolecules having reactive groups, providing at least two molecules of a linking agent, providing a spacing agent, reacting the linking agent, the spacing agent and the biomolecules, so that the biodegradable cross-linking moiety is formed between the biomolecules to provide cross-linking and thereby make the implantable bioprosthesis.

Further provided is a method of using the implantable bioprosthesis of the invention, the method comprising providing the implantable bioprosthesis, providing an organism comprising an enzyme capable of degrading the spacer, contacting the implantable bioprosthesis with the enzyme, and degrading the implantable bioprosthesis and thereby permitting expansion of the implantable bioprosthesis. In certain embodiments of the method, degrading of the implantable bioprosthesis proceeds at the biodegradation rate and wherein the biodegradation rate is affected by the amount of the spacer.

### BRIEF DESCRIPTION OF SEVERAL VIEWS OF THE DRAWINGS

The invention will be described in conjunction with the following drawings in which like reference numerals designate like elements and wherein:

FIG. 1 is a scheme, which depicts cross-linking of collagen with a biodegradable cross-linking moiety formed by reacting triglycidyl amine (TGA) and cystamine, wherein XH depicts any nucleophilic group of amino acid residue capable of epoxy-ring opening (NH2 of lysine, S⁺(H)CH₃ of methionine, imidazolyl of histidne, etc.

FIG. 2 is a scheme, which depicts synthesis of triglycidyl amine (TGA).

FIG. 3 is a scheme, which depicts synthesis of N,N′-triglycidyl 1,3-diaminopropane.

FIG. 4 is a bar graph demonstrating structural strength of bovine pericardium after crosslinkage with TGA or with TGA and cystamine. Structural strength of bovine pericardium, as estimated by the shrink temperatures obtained by Differential Scanning Calorimetry (DSC) is not decreased by the inclusion of cystamine (2-100 mM) during crosslinking with TGA.

FIG. 5 is a series of brightfield photomicrographs of two types of cultures such as sheel aortic valve interstitial cells (SAVIC) and rat vascular smooth muscle cells (A10) on different collagen substrates such as native collagen, collagen-100 mM TGA, collagen-100 mM TGA/50 mM cystamine, and collagen-100 mM TGA/100 mM cystamine using 100× magnification. Cell morphology after 24 hours of culture on modified collagen substrates shows that the combination of TGA with cystamine does not interfere with the biocompatibility of TGA crosslinked collagen.

FIG. 6 is a series of brightfield photomicrographs of two types of cultures on different collagen substrates as shown in FIG. 5 further treated with Alizarin Red to detect calcification. After 14 days in culture, slight cell aggregation was observed in SAVIC cultures growing with 100 mM cystamine, but no calcification was detected by Alizarin Red staining. Gross calcification was observed in the absence of TGA. The brightfield photomicrographs are shown using 100× magnification.

FIG. 7 is a bar graph demonstrating biodegradation of cultured substrates after 14 days in M199/10% FBS as measured by % of weight loss of recovered substrate gels. Weight loss was calculated by comparison to native collagen gels incubated without cells. All calculations were performed in triplicate. It was observed that A10 cells digest the culture matrix to a lesser degree than SAVIC cells. Further, the addition of cystamine increases biodegradation in a dose-dependent manner.

FIG. 8 is a diagram, which depicts polymerization of triglycidyl amine in solution. A is a non-reactive, biocompatible anion.

### DETAILED DESCRIPTION OF THE INVENTION

The invention was driven by the desire to develop an implantable bioprosthesis capable of cell mediated remodeling via controlled enzymatic degradation once the bioprosthesis is implanted in an organism or otherwise subjected to enzymes. Enzymatic degradation of the bioprosthesis causes cell ingrowth and consequently allows cell mediated remodeling of the bioprosthesis. Thus, the implantable bioprosthesis of the invention comprises biodegradably and artificially cross-linked biomolecules.

An implantable bioprosthesis comprising proteins treated with a polyepoxy amine compound is described in U.S. Pat. No. 6,824,970 to Vyavahare et al, incorporated herein in its entirety.

As described in U.S. Pat. No. 6,824,970, the treatment of the implantable bioprosthesis with triglycidyl amine (TGA) confered material stability, resistance to calcification, and biocompatibility to the implantable bioprosthesis. However, TGA based crosslinks are epoxy based and therefore are irreversible. Thus, although TGA crosslinked heterografts support cell overgrowth, no cell ingrowth remodeling of the TGA-crosslinked extracellular matrix (ECM) has occurred. This poses a disadvantage for use, for example, with tissue engineering strategies in situations where relative growth of the implant would be desirable, e.g. a heart valve prosthesis implanted in a young child. The present invention presents a strategy for formulation that can be degraded through cellular interactions. The desired formulation involves using a biodegradable intermediary, such as a biodegradable spacer, which permits cell mediated remodeling to bridge between cross-links, for example, TGA cross-links. As a non-limiting example, the desired formulation involves TGA crosslinking in the presence of cystamine, which permitted stable TGA crosslinking (as evidenced by shrink temperatures), but showed biodegradation in cell culture.

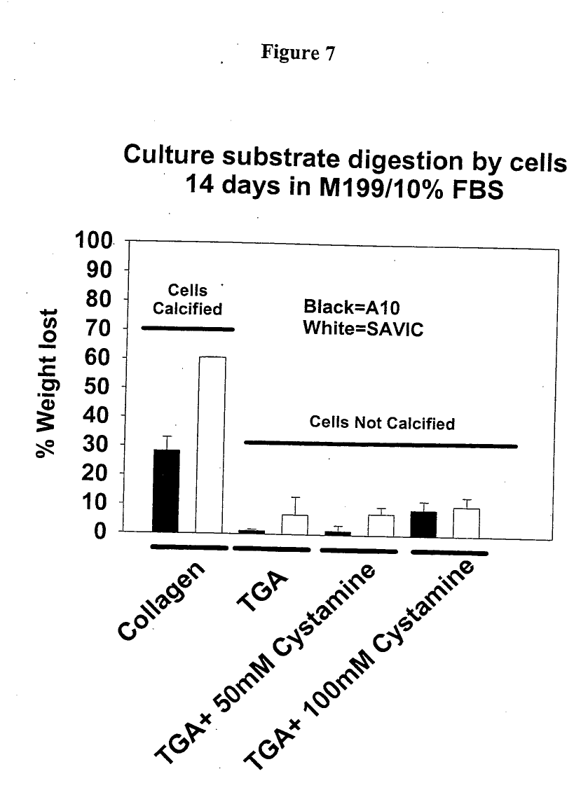

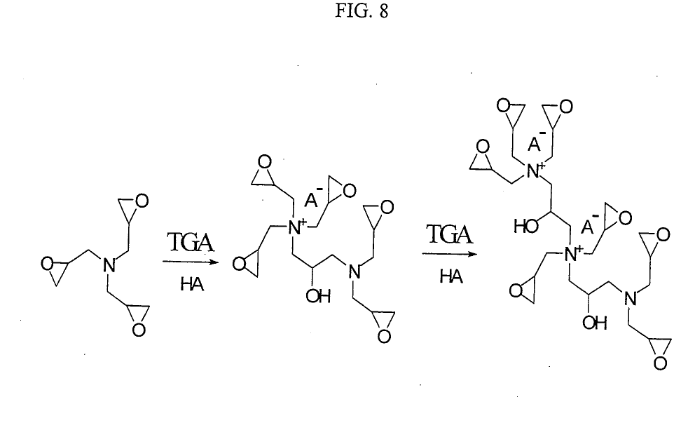

The implantable bioprosthesis of the invention comprises biomolecules having a reactive moiety and optionally a reactive group, and a biodegradable cross-linking moiety having (a) at least two linking moieties, wherein the at least two linking moieties are non-biodegradable and (b) a spacer, wherein the spacer is biodegradable and is in communication with the at least two linking moieties, provided that the biodegradable cross-linking moiety is artificial and is covalently bound to the reactive moiety, whereby the biomolecules are cross-linked. In certain embodiments, the rate of degradation is controlled, wherein the implantable bioprosthesis is adapted to sufficiently degrade upon exposure to a cell or an enzyme at a biodegradation rate to permit an expansion of the implantable bioprosthesis and wherein the biodegradation rate is affected by an amount of the spacer. As shown in Example 6 and FIG. 7, the biodegradation rate can be increased, for example, by increasing the amount of biodegradable spacer, cystamine, or by selecting cells that degrade the spacer at a higher rate.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Artificially” cross-linked biomolecules are biomolecules that are not cross-linked by the presence of naturally occurring cross-links.

A “bioprosthesis” is an implantable protein-containing article, all or part of which comprises a biological tissue (e.g. a tissue obtained from an animal or a cultured animal tissue, such as tissue obtained from a human or a cultured human tissue), a component of such a tissue (e.g. cells or extracellular matrix of the tissue), or some combination of these. Bioprosthesis specifically retain and enable biologic structure and function in their intended implant configuration. Examples of bioprostheses or components include, but are not limited to, an artificial heart, a heart valve prosthesis, an annuloplasty ring, a dermal graft, a vascular graft, a vascular, cardiovascular, or structural stent, a vascular or cardiovascular shunt, a dura mater graft, a cartilage graft, a cartilage implant, a pericardium graft, a ligament prosthesis, a tendon prosthesis, a urinary bladder prosthesis, a pledget, a suture, a permanently in-dwelling percutaneous device, an artificial joint, an artificial limb, a bionic construct (i.e. one of these bioprostheses comprising a microprocessor or other electronic component), a coated stent, a coated catheter, a synthetic analog of a bioprosthetic tissue, and a surgical patch.

A “derivative” refers to chemical substance derived from another substance either directly or by modification or partial substitution.

“Implantation,” and grammatical forms thereof, refers to the process of contacting a prosthesis (e.g. a bioprosthesis) with a tissue of an animal in vivo wherein the contact is intended to continue for a period of hours, days, weeks, months, or years. Such contact includes, for example, grafting or adhering the prosthesis to or within a tissue of the animal and depositing the prosthesis within an orifice, cavity, incision, or other natural or artificially-created void in the body of the animal.

A “reactive moiety” is portion of a reactive group of a biomolecule, which is remaining after the reactive group has reacted with a linking group.

A “biodegradable cross-linking moiety” is an artificial moiety capable of enzymatic degradation which is formed by reacting the linking agent and the spacing agent with reactive moieties and/or reactive groups of biomolecues to cross-link biomolecules of the bioprosthesis.

A “linking agent” as used herein is a non-biodegradable substance, which is capable of reacting with reacting groups of biomolecules to form non-biodegradable crosslinks.

A “linking moiety” as used herein is a non-biodegradable moiety, which is a portion or a residue of the linking agent. The linking moiety cross-links biomolecules to form non-biodegradable crosslinks. A combination of at least two linking moieties and at least one biodegradable spacer forms the biodegradable cross-linking moiety and provides biodegradable crosslinks.

A “spacing agent” is a biodegradable substance, which is capable of (1) enzymatic degradation and (2) reacting with the linking agent.

A “spacer” is biodegradable moiety, which is a portion of a spacing agent covalently bound to the linking moiety after reacting the spacing agent and the linking agent.

“Enzymatic degradation” as used herein is breaking of cross-linking bonds caused by enzymes, for example, breaking of disulfide bonds by disulfide reductases. Enzymes are produced by cells such as, for example, SAVIC cells or A10 cells or can be genetically engineered to be produced by organisms not normally producing such enzymes. For example, a gene therapy vector expressing a disulfide reductase could be administered locally to the crosslinked area for controlled biodegradation. Disulfide reductases are a major class of enzymes responsible for reducing or degrading disulfide bonds. Thioredoxins with a dithiol/disulfide active site (CGPC) are the major cellular protein disulfide reductases.

Glutaredoxins catalyze glutathione-disulfide oxidoreductions overlapping the functions of thioredoxins and using electrons from NADPH via glutathione reductase. All mammalian thioredoxin reductase isozymes are homologous to glutathione reductase and contain a conserved C-terminal elongation with a cysteine-selenocysteine sequence forming a redox-active selenenylsulfide/selenolthiol active site and are inhibited by goldthioglucose (aurothioglucose) and other clinically used drugs.

“Stabilization” and grammatical forms thereof of a bioprosthesis means increasing the mechanical strength of the bioprosthesis, decreasing the rate or incidence of degradation of the bioprosthesis following its implantation in or on an animal, or some combination of these. Causes of degradation include mechanical wear, reactions between the prosthesis and the animal's immune system, and calcification associated with the prosthesis. Stabilization can enhance one or more of the durability, shelf life, and fatigue life of the bioprosthesis. Exemplary means of stabilizing a bioprosthesis include covalently linking (“cross-linking”) components (e.g. proteins) of the prosthesis, inhibiting calcification associated with the prosthesis, co-incorporating a beneficial polymer or another agent into the bioprosthesis, and stabilizing a glycosaminoglycan (GAG) on the prosthesis or a tissue associated with the prosthesis. For example, a GAG is “stabilized” on a tissue when the tissue is reacted with a reagent, which generates at least two covalent bonds associated with a GAG molecule. These bonds can either be intramolecular or intermolecular in nature. A reagent, which generates such bonds is herein designated a “GAG-stabilizing reagent”. The terms “stabilization,” “fixation,” and “cross-linking” are used interchangeably herein.

A GAG or protein is “endogenous” with respect to a tissue if the GAG or protein is normally present on or in the tissue in a healthy individual which naturally comprises the tissue (e.g. a GAG or protein which naturally occurs on or in the tissue, regardless of whether the GAG or protein was isolated with the tissue or was added to the tissue after isolation thereof). Otherwise, the GAG or protein is “exogenous” with respect to the tissue.

A “polyepoxy amine” is a chemical species containing both at least one amine moiety (e.g. a primary, secondary, tertiary, or quaternary amine moiety, such as an oligomer of triglycidyl amine) and a plurality of epoxide moieties. Polyepoxy amines in this group include, for example, diepoxy amines and triepoxy amines. The polyepoxy amine compound also comprises a carbon-containing moiety (e.g. an alkyl group such as a C₁-C₆ straight chain alkyl group) interposed between an epoxy moiety and an amine moiety of the compound.

An “epoxy-reactive moiety” is a moiety capable of reacting with an epoxide ring such that the epoxide ring is opened and a covalent bond is formed between the moiety and an atom of the epoxide ring.

Bioprostheses

Bioprostheses comprise biomolecules having a plurality of reactive groups. Preferably, the reactive groups are epoxy reactive groups. In certain embodiments, the biomolecules are derived from a biological tissue or a synthetic analog of a bioprosthetic tissue or both. Pre-implantation treatment with the linking agent and the spacing agent can be used to stabilize bioprostheses such as artificial hearts, heart valve prostheses, vascular grafts, annuloplasty rings, dermal grafts, dura mater grafts, pericardium grafts, cartilage grafts or implants, pericardium grafts, ligament prostheses, tendon prostheses, urinary bladder prostheses, pledgets, sutures, permanently in-dwelling percutaneous devices, surgical patches, vascular, cardiovascular, or structural stents, coated stents and catheters, vascular or cardiovascular shunts, and the like. Preferably, the bioprosthetic device is a heart valve prosthesis. Biological tissue treated with a polyepoxy amine compound prior to implantation can be obtained from the recipient, from an animal of the same species as the recipient, or from an animal of a different species than the recipient. Exemplary donor animals include mammals such as humans, cows, pigs, dogs, and seals, and kangaroos. Exemplary tissues include hearts, heart valves, aortic roots, aortic wall, aortic leaflets, pericardial tissues (e.g. pericardial patches), connective tissues, dura mater, bypass grafts, tendons, ligaments, dermal tissues (e.g. skin), blood vessels, umbilical tissues, bone tissues, fasciae, and submucosal tissues. The tissue can, alternatively, be a cultured tissue, a prosthesis containing extracellular matrix obtained from an animal, a reconstituted tissue (e.g. bone cells in an artificial bone-like medium), or the like. In certain embodiments, the biomolecules are members selected from the group consisting of proteins, glucosoaminoglucans, and an extracellular matrix. The stabilization method described herein can also be used to stabilize bioprostheses comprising one or more materials of non-biological origin, wherein the material has surface chemical groups like those of a biological tissue (e.g. as with a protein-containing synthetic matrix).

Synthetic analogs of bioprosthetic tissues can be formed from synthetic polymers, biological polymers, or both, including those generally found in natural tissue matrices. Suitable synthetic polymers include, for example, polyamides and polysulfones. Biological polymers can be naturally-occurring or produced in vitro by, for example, fermentation and the like. Purified biological polymers can be appropriately formed into a substrate by techniques such as weaving, knitting, casting, molding, extrusion, cellular alignment, and magnetic alignment. Suitable biological polymers include, without limitation, collagen, elastin, silk, keratin, gelatin, polyamino acids, polysaccharides (e.g. cellulose and starch), and copolymers of any of these. For example, collagen and elastin polymers can be formed into a synthetic bioprosthetic tissue analog by any of a variety of techniques, such as weaving and molding. Synthetic tissue analogs mimic a natural tissue matrix. Alternatively, synthetic substrates can be used to form a tissue analog, either alone or together with naturally-occurring substrates. Synthetic tissue analogs can be implanted with or without cells seeded on or within them. Such tissue analogs can, optionally, be resorbable.

The reactive moiety which is formed upon reaction of a reactive group with a linking agent can, for example, be a methylthio moiety (e.g. the methylthio moiety of a methionine residue in a protein), a primary amine moiety (e.g. the primary amine moiety of a lysine residue in a protein), a phenolic hydroxyl moiety (e.g. the hydroxyl moiety of a tyrosine residue in a protein), a phosphate moiety (e.g. the phosphate moiety of a phosphatidyl serine residue in a protein), or a carboxyl moiety (e.g. the carboxyl residue of a glutamate residue in a protein). If more than one reactive group is reacted, the reactive moiety can be secondary, tertiary, or quaternary moiety, such as, for example, secondary, tertiary, or quaternary amine moiety.

In certain embodiments, the reactive group is a member selected from the group consisting of an amine group, a hydroxyl group, a phosphate group, and a carboxyl group. In certain embodiments, the reactive moiety is a member selected from the group consisting of an amine moiety, a hydroxyl moiety, a derivative of a phosphate moiety, and a carboxyl moiety.

The invention includes stabilized, implantable biodegradable bioprostheses, which have a plurality of epoxy-reactive moieties, which have been reacted with a linking agent represented by at least one of polyepoxy amine, aldehyde, and carbodidimide and a spacing agent (i.e. implantable bioprostheses having epoxy-reactive moieties which are cross-linked with one or more biodegradable cross-linking moiety). For example, all, substantially all, or a fraction (e.g. 90%, 80%, 70%, 50%, 25%, 10%, 5%, or 1% or fewer) of surface epoxy-reactive moieties of the bioprosthesis can be cross-linked with one or more biodegradable cross-linking moiety.

The bioprostheses of the invention and methods described herein are not limited to those which include a polyepoxy amine compound as a preferred linking agent. Other agents, such as additional linking agents, calcification inhibitors, GAG-stabilizing agents, and the like can be used in conjunction with (i.e. before, during, or after) polyepoxy amine treatment.

The invention further includes implantable prosthesises comprising at least a polyepoxy amine and, optionally, one or more of a buffering agent, a physiological salt (e.g. NaCl and KCl), glycosaminoglycan, a glycosaminoglycan-stabilizing reagent, a second linking agent, and a calcification inhibitor. These compositions can further comprise aqueous and non-aqueous liquids, blood or blood products, and other liquids which facilitate processing of a bioprosthesis using a polyepoxy amine compound as described herein.

In certain embodiments, the implantable bioprosthesis further comprises at least one of a calcification inhibiting moiety or a glucosoaminoglycan stabilizing moiety, wherein the calcification inhibiting moiety is formed by treating the implantable bioprosthesis with a calcification inhibitor and the glucosoaminoglycan stabilizing moiety is formed by treating the implantable bioprosthesis with a glucosoaminoglycan stabilizing reagent. In certain embodiments, the calcification inhibitor is a member selected from the group consisting of an Al⁺³ salt, a Fe⁺³ salt, and aminobisphosphonate.

Stabilization of a bioprosthetic tissue can be enhanced by stabilizing GAGs, which occurs endogenously in the tissue. Examples of GAG-stabilizing reagents include carbodiimides such as 1-ethyl-3-(3dimethyl-aminopropyl)carbodiimide (EDAC), heterofunctional azides, and carbohydrate-protein linking reagents. Combined use of a GAG-stabilizing reagent and a polyepoxy amine compound to treat an implantable bioprosthetic device can enhance stabilization of the bioprosthesis relative to treatment with the polyepoxy amine compound alone. Therefore, the invention encompasses using a polyepoxy amine compound and a GAG-stabilizing reagent, either simultaneously or sequentially in either order, for stabilization of a bioprosthetic tissue. The GAG, which is stabilized in and on the bioprosthesis can be either endogenous or exogenous. The spacing agent can be added prior to the addition of the GAG-stabilizing reagents, after the addition or simultaneously with the addition.

In certain embodiments, the implantable bioprosthesis further comprises a non-biodegradable cross-linking moiety having the at least one linking moiety, provided that the non-biodegradable cross-linking moiety is free of the spacer and is covalently bound to the reactive moiety. The non-biodegradable cross-linking moiety is a cross-linking moiety, which does not include the biodegradable spacer. In certain embodiments, the biodegradable cross-linking moiety and the non-biodegradable cross-linking moiety are at a ratio of about 1 to about 10.

The inventors had at least two objectives in creating the present implantable prosthesis: one objective was to stabilize a bioprosthesis and another objective was to confer the ability to biodegrade to the bioprosthesis. The biodegradable cross-linking moiety having at least two linking moieties and a biodegradable spacer combines both objectives.

Biodegradable Cross-Linking Moiety

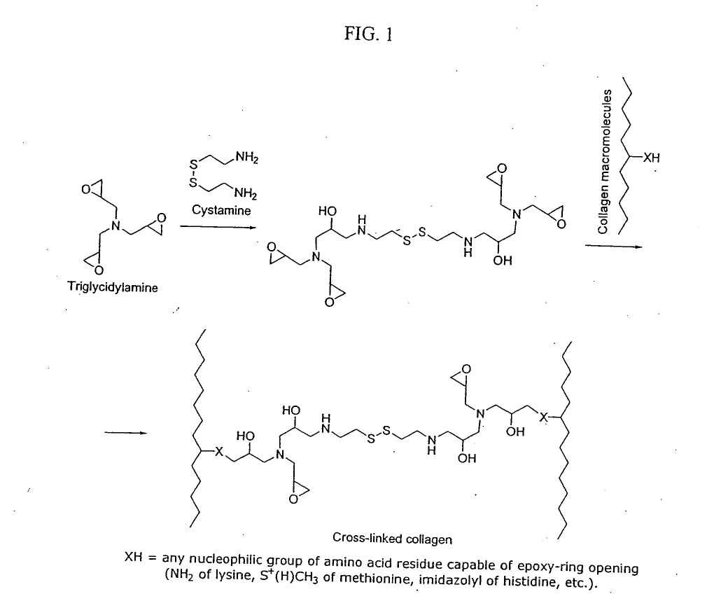

The biodegradable cross-linking moiety is formed by reacting the linking agent and the spacing agent. The biodegradable cross-linking moiety comprises the linking moiety and the spacer. A non-limiting example of reacting TGA and cystamine is shown in FIG. 1. In this reaction, the biodegradable cross-linking moiety is represented by the formula: 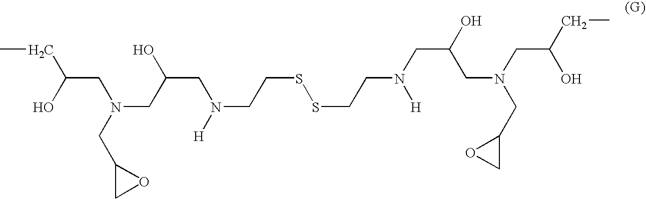
The Linking Moiety and the Linking Agent

The stabilization is effected by contacting a bioprosthesis with a linking agent, for example, aldehyde (e.g., glutaraldehyde), carbodidimides, polyepoxy ethers or polyepoxy amine (as described in U.S. Pat. Nos. 6,824,970 and 6,391,538), preferably in the presence of an aqueous liquid, to yield an implantable bioprosthesis comprising proteins cross-linked with one or more linking moieties. Glutaraldehyde is an effective and widely used cross-linker, but, as discussed above, its use can lead to unacceptable levels of calcification in the bioprosthesis. Other cross-linking reagents which do not necessarily react with the bioprosthesis itself include, for example, chain extenders and dicarboxylic acids. A polyepoxy amine compound can be used alone to provide adequate cross-linking. However, a greater degree of cross-linking can further enhance the mechanical stability of the prosthesis. Therefore, it can be advantageous to use other cross-linking reagents in conjunction with a polyepoxy amine compound in order to obtain a higher degree of cross-linking in the bioprosthesis. Accordingly, the present invention includes using a polyepoxy amine compound alone or in combination with one or more other linking agents.

The polyepoxy amine can form covalent bonds between chemical groups on or within the bioprosthesis, including amine groups (e.g. primary, secondary, and tertiary amine groups), thio groups (e.g. thiol and methylthio groups), hydroxyl groups (e.g. phenolic and carboxylic hydroxyl groups of tyrosine, aspartate, glutamate side chains in a protein) and phosphate groups. As a result of contacting the bioprosthesis with the polyepoxy amine, covalent chemical bonds are formed between the polyepoxy amine and the bioprosthesis, such as bonds between a polyepoxy amine and one or more amino acid residue side chains (e.g. side chains of one or more proteins, including both proteins which are endogenous to a tissue of the bioprosthesis and exogenous proteins), between one molecule of a polyepoxy amine compound and another molecule of the same polyepoxy amine compound, or between both an amino acid residue side chain and another molecule of the polyepoxy amine. Using a polyepoxy amine results in formation of covalent linkages between epoxy-reactive moieties of the bioprosthesis. Thus, a network of inter-connected chemical groups of the bioprosthesis (e.g. interconnected amino acid residue side chains) is generated, thereby stabilizing the bioprosthesis. If the bioprosthesis is contacted with a polyepoxy amine compound in the presence of another compound (e.g. a protein which does not normally occur in the bioprosthesis), then the other compound can be linked to the bioprosthesis.

In certain embodiments, at least one of the at least two linking moieties comprises an amine moiety and a hydroxyl group formed by a ring-opening reaction of an epoxide. In certain embodiments, at least one of the at least two linking moieties can be selected from derivative of polyepoxy amine and a derivative of aldehyde. Derivatives as used herein include residues of polyepoxy amines and aldehydes formed after reacting.

In certain embodiments, the derivative of polyepoxy amine is a member selected from the group consisting of

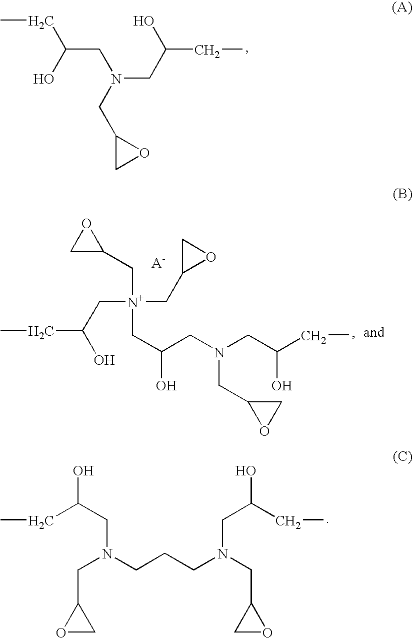

In certain embodiments, the derivative of polyepoxy amine is a derivative of triglycidyl amine or N,N′-Tetraglycidyl-1,3-diaminopropane. As shown in (B), A⁻ denotes an anion of an acid present in the reaction, such as, for example, dihydrophosphate (H₂PO₄⁻) and hydrophosphate (HPO₄⁻) anions.

In another embodiment, the polyepoxy amine is a polymer having a plurality of epoxide groups attached thereto (e.g. at one or both ends or as side chains within or throughout the polymer). It is recognized that some polyepoxy amines have an epoxy-reactive moiety (e.g. a non-quaternary amine moiety) and can undergo autopolymerization, giving rise to linear or branched polymers. The length or degree of branching can be controlled by, for example, modulating the length of time the polyepoxy amine preparation is permitted to autopolymerize. For example, TGA is a tertiary amine, which can autopolymerize, as shown in FIG. 8.

In FIG. 8, the abbreviation HA denotes any acid present in the solution, including, for example, dihydrophosphate (H₂PO₄⁻) and hydrophosphate (HPO₄⁻) anions. If no acid is present, H₂O acts like an acid (the anion is OH⁻ in this case). Situations when no anion is formed are also contemplated in the invention, e.g., when the acid is R₃NH⁺.

When such a polymer is used, the polymer can be formed by polymerizing polyepoxy amine compound molecules with a polyepoxy amine compound molecule that is already bound with one or more moieties of the bioprosthesis, by polymerizing the polyepoxy amine compound prior to contacting it with the bioprosthesis, or both. When a polyepoxy amine polymer is used, the polymer can be a linear polymer or a branched polymer, and preferably has a molecular weight of about 185 to 10,000. For example, a polymer of TGA can be used in which the polymer is formed by polymerization of at least about 15 TGA molecules, yielding a TGA polymer having a molecular weight greater than 3000. As indicated in FIG. 8, polymerization of a polyepoxy amine compound can lead to formation of a polymer having a plurality of quaternary ammonium moieties. Prior art polyepoxide compounds (e.g. Denacol™ products) do not contain amino groups, and thus do not auto-polymerize to form polyepoxy amine compound polymers having quaternary ammonium groups. While not being bound by any particular theory of operation, it is believed that the quaternary ammonium moieties in the polyepoxy amine compound polymers described herein are, at least in part, responsible for the improved stabilization properties of bioprostheses treated with such polymers.

In addition to covalently linking chemical moieties of a bioprosthesis, treatment of a bioprosthesis with a polyepoxy amine compound inhibits post-implantation calcification associated with the prosthesis. Examples of prior art calcification inhibitors include ethanol, aminobisphosphonate, Al⁺³ salts (e.g., aluminum chloride), Fe⁺³ salts, chondroitin sulfate, propylene glycol, alpha amino oleic acid, surfactants, detergents, and the like. In contrast to these compounds, which require additional reagents to substantially enhance the mechanical durability of bioprostheses with which they are contacted, polyepoxy amine compounds, unaccompanied by additional reagents, can both covalently link chemical moieties and substantially improve calcification resistance in the bioprosthesis. As demonstrated in Example 6, addition of the biodegradable spacer does not inhibit calcification resistance in the bioprosthesis.

Polyepoxy amine can be prepared using synthetic methods known in the art (e.g., by a modification of a method described in Ross et al., 1963 J. Org. Chem. 29:824-826, or as described in Martyanova et al., 1990, Sb. Nauch. Tr. Lenengr. In-tKinoinzh. 2:139-141 (Chem. Abst. Nos. 116:43416 and 116:31137) or Chezlov et al., 1990, Zh. Prikl. Khim. (Leningrad) 63:1877-1878 (Chem. Abst. No. 114:121880)).

Biodegradable Spacer and Spacing Agent

Biodegradability is conferred to the bioprosthesis by crosslinking the biomolecules with the biodegradable cross-linking moiety comprising a biodegradable spacer. The rate of biodegradation is controlled by the amount of the spacer and also by the availability of enzymes capable of degrading the spacer.

In certain embodiments, the spacer comprises a disulfide group or a carbonyl group. In certain embodiments, the spacer is represented by a formula: 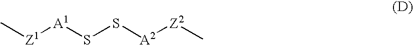
wherein A¹ and A² each comprise at least one carbon atom, and Z¹ and Z² are nucleophilic groups capable of opening an epoxy ring. In certain embodiments, at least one of the nucleophilic groups is a member selected from the group consisting of an amino group, an alkylthio group, a derivative of imidazole, a derivative of pyrazole, and a derivative of pyridine. A derivative of imidazole, pyrazole, and pyridine as used herein means a residue of respective substances.

In certain embodiments, the spacer is represented by a formula:
—NH—(CH₂)₂—S—S—(CH₂)₂—NH— (E)

In certain embodiments, the spacer is represented by a formula:

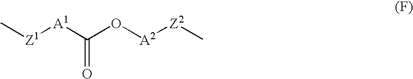

The spacer as a part of the biodegradable cross-linking moiety is formed by reacting the linking agent and the spacing agent. In certain embodiments of the method, the spacing agent is represented by a formula: 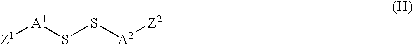
wherein A¹ and A² each comprise at least one carbon atom, and Z¹ and Z² are nucleophilic groups capable of opening an epoxy ring. In certain embodiments, the spacing agent is cystamine.

In certain embodiments of the method, the spacing agent is represented by a formula: 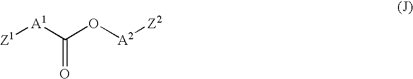
Method of Making an Implantable Bioprosthesis

Further provided is a method of making an implantable bioprosthesis of the invention, the method comprising providing an untreated bioprosthesis comprising biomolecules having reactive groups, providing at least two molecules of a linking agent, providing a spacing agent, reacting the linking agent, the spacing agent and the biomolecules, so that the biodegradable cross-linking moiety is formed between the biomolecules to provide cross-linking. A non-limiting example of the method is shown in FIG. 1 using TGA as the linking agent and cystamine as the spacing agent.

The spacing agent can be used at various concentrations to provide the desired biodegradable rate. As a non-limiting example, cystamine was used at 2-100 mM; it was observed that 100 mM provided higher degradation that 50 mM. The spacing agent can be provided as a solution or solid and combined with the linking agent during the reaction. The additional amounts of the spacing agent can be added subsequently or prior to adding the linking reagent.

The linking agent can be used at substantially any concentration. However, it is preferred that the concentration of the linking agent be high enough to yield an appreciable rate of reaction. For example, the rate of reaction can be such that a degree of cross-linking not less than about 50% that achievable using glutaraldehyde (i.e. as assessed, for example by determining the thermal shrinkage temperature using, e.g., differential scanning calorimetry) is attained within not more than about 30 days, and preferably within not more than 10 days of reaction. By way of example, when the linking agent is triglycidyl amine, a preferred range of concentrations is about 0.01 to 1 Mr, and is preferably about 100 mM for the reaction conditions described herein.

The aqueous liquid used in the stabilization method should, especially for tissue-containing bioprostheses, maintain the pH of the stabilization reaction mixture at about 5 to about 11, preferably 6-10, more preferably 7-10, and most preferably about 7.0-7.4. Nonetheless, the stabilization method described herein can be used even at lower pH values. When the bioprosthesis comprises (or is entirely made from) a synthetic material, the stabilization reaction can be performed within an even broader pH range, such as at a pH of about 2-12. The liquid can, for example, be a buffer, such as 10-500 mM sodium or potassium HEPES buffer at a pH of about 6.9 to 7.9 or 10-500 mM sodium or potassium borate buffer at a pH of about 8.5 to 9.5. The buffer is preferably used in excess, relative to the amount of a polyepoxy amine compound that is present, or even relative to the average number of epoxy moieties per molecule of the polyepoxy amine compound multiplied by the amount of polyepoxy amine compound. As an alternative to using an excess of buffer, a reaction mixture having a lower amount of buffer can be used if the mixture is replaced with fresh (i.e. non-reacted) reaction mixture from time to time. In this alternative, the pH of the reaction mixture can be monitored, and the reaction mixture can be replaced or supplemented with additional buffer upon detection of a significant pH change. The buffer should be selected such that it does not chemically react with the polyepoxy amine compound or the bioprosthesis in a manner that would inhibit reaction of the polyepoxy amine compound with the bioprosthesis. Most common buffers (e.g. borate, HEPES, carbonate, cacodylate, citrate, TRIS, and MOPS) are suitable for use. The incorporation of the buffer into tissues can occur during the reaction.

The pH of the stabilization reaction mixture can increase as the reaction between the polyepoxy amine compound and the bioprosthesis proceeds. Preferably, the pH of the reaction mixture is maintained at or below a maximum value (e.g. at or below pH 10, preferably at or below 7.4). pH maintenance can be achieved by any known method, including acidification of the mixture, addition of buffer, and replacement of the reaction mixture with fresh reaction mixture having a desirable pH (e.g. pH 7-7.4). For example, the reaction mixture can comprise 100 mM HEPES and 100 mM TGA, can have a pH of about 7.0, and can be replaced on a daily basis.

The duration of the period during which the bioprosthesis and the polyepoxy amine compound are maintained in contact can vary from about 3 hours to several months or longer. The duration of contact is preferably at least about 3 days, and more preferably, at least about 7 days. Contacting the bioprosthesis and the polyepoxy amine compound in an aqueous liquid for 8-10 days is considered sufficient.

The temperature of the stabilization mixture can be substantially any temperature at which the reaction can proceed at an appreciable rate and at which the bioprosthesis is not damaged. It is understood that the rate of reaction increases with increasing temperature. If the bioprosthesis contains protein (e.g. if it comprises a tissue obtained from an animal), then the temperature of the reaction mixture can be maintained, for example, at 20-37° C.

Further provided is a method of using the implantable bioprosthesis of the invention, the method includes providing the implantable bioprosthesis, providing an organism comprising an enzyme capable of degrading the spacer, contacting the implantable bioprosthesis with the enzyme, and degrading the implantable bioprosthesis and thereby permitting expansion of the implantable bioprosthesis. In certain embodiments of the method, degrading of the implantable bioprosthesis proceeds at the biodegradation rate and is affected by the amount of the spacer.

The invention will be illustrated in more detail with reference to the following Examples, but it should be understood that the present invention is not deemed to be limited thereto.

### EXAMPLES

### Example 1

### Synthesis of TGA

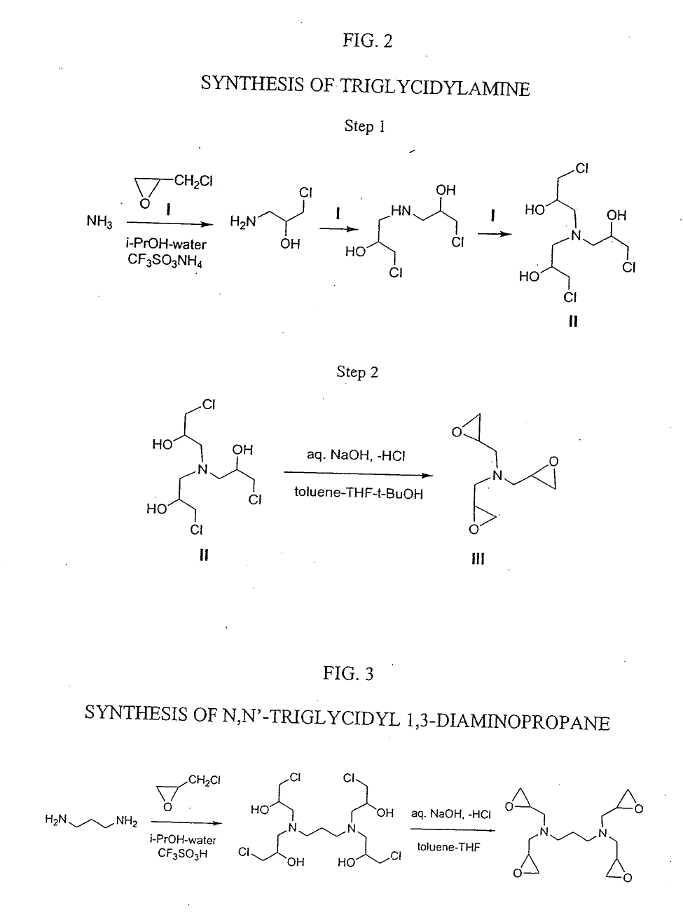

One method of preparing TGA is disclosed in U.S. Pat. No. 6,391,538. In this invention, several modifications were introduced into the procedure as compared to the TGA synthesis disclosed in U.S. Pat. No. 6,391,538 to facilitate the transition to large-scale preparations, however, the disclosed method is also applicable. The synthesis is described in detail below. As shown in FIG. 2, triglycidylamine (TGA) (III) was prepared using a two-step procedure. In step 1, ammonia was reacted with epichlorohydrin (I) in aqueous isopropanol in the presence of ammonium triflate as a catalyst to give tris-(3-chloro-2-hydroxypropyl)amine (II). In step 2, the latter was dissolved in a mixture of solvents (toluene, tetrahydrofuran and tert-butanol) and dehydrochlorinated by addition of concentrated aqueous NaOH, forming a substance (III) with the epoxy-ring closure.

### Step 1. Preparation of tris-(3-chloro-2-hydroxypropyl)amine (II)

Aqueous 29% ammonia (d=0.895, 55 mL, 0.84 mol) was added to a mixture of epichlorohydrin (315 mL, 4.03 mol), isopropanol (300 mL) and ammonium triflate (1.67 g, 10 mmol). The mixture was allowed to react in a three-necked 1 L flask with magnetic stirring at 20-24° C. for 48 hours; a water bath with cool water (16-20° C.) was applied in the first 8 hours of reaction to prevent overheating. The reaction solution was then stirred at 30-35° C. (inside temperature) for 3 hours to complete the reaction and transferred into a 2 L round bottom flask. The mixture was carefully dried on a rotary evaporator (RE) at 30° C. (in the beginning) to 40° C. (in the end) and 15-20 mm Hg up to the end of distillation. To completely remove the excess of epichlorohydrin, the resulting thick syrup (about 300 g) was dissolved in toluene (250 mL) with addition of tert-butanol (70 mL), and the solution was dried on RE at 40-45° C. and 15-20 mm Hg up to the end of distillation (weight of syrup was about 290 g). The co-evaporation with toluene (250 mL) and tert-butanol (70 mL) was repeated using the same conditions. Tris-(3-chloro-2-hydroxypropyl)amine (II) was obtained as a thick colorless syrup (293 g) and used in the Step 2 without any additional purification.

### Step 2. Preparation of Crude Triglycidylamine (III)

Tris-(3-chloro-2-hydroxypropyl)amine (II) (syrup, 293 g) was transferred into a 2L three-necked flask with tetrahydrofuran (80 mL) and tert-butanol (15 ml, Note 7) and diluted with toluene (450 mL). The reaction flask was provided with a powerful mechanical stirrer, a thermometer and a dropping funnel. A solution of NaOH (296 g, 7.4 mol) in water (296 mL) was added to the mixture in 0.5 h at 18-22° C. under vigorous stirring and cooling on an ice-water bath. The mixture was additionally stirred at the same temperature for 2 h (no external cooling was necessary) and diluted with ice-cold water (460 mL). During the dilution, the temperature remained below 25° C. without any external cooling. The mixture was additionally diluted with toluene (100 mL), stirred for 5 min. and transferred into a 2L separating funnel. The organic layer was separated, the alkaline layer (containing about 10 g of a semi-solid polymer) was extracted with toluene (150 mL), and the organic phases were separately dried overnight over anhydrous potassium carbonate at 4° C. The desiccant was filtered off and thoroughly washed with toluene (about 80 mL), the filtrate was vacuum-concentrated on RE at the vacuum from 15-20 mm to 1 mm Hg and the temperature up to 50° C. to give crude triglycidylamine (III) as an almost colorless viscous oil (about 150 g).

### Vacuum-Distillation of Crude Triglycidylamine (III)

The crude triglycidylamine (III) (150 g) was vacuum-distilled at 0.08-0.10 mm Hg without fractionation to give 115.5 g (74%, calculated for NH3) of distilled product, bp. 75-95° C., almost pure on thin-layered chromatography (TLC). This product was fractionated at 0.08 mm Hg to give 112 g (72%) of pure triglycidylamine (111), bp. 80-85° C. Viscous liquid, slowly solidifying at −15° C., and then remaining solid at room temperature was obtained.

The following considerations are important. During the second day and most of the first day, the reaction mixture needs no supervision. Since solutions of ammonia are prone to decrease the concentration during the storage, the concentration of aqueous ammonia was thoroughly checked by e.g., measuring the density. The presence of ammonium triflate prevents spontaneous self-acceleration of the reaction; to diminish the loss of NH₃ in the beginning, the flask was connected to the atmosphere via a narrow tube with a cotton plug. When the unreacted ammonia was still present in the reaction mixture, it was unreasonable to increase the temperature otherwise side reactions could take place. The optimal reaction time at room temperature may be less than 48 h, however after 18 h some ammonia still can be detected.

The mixture is prone to foaming, and measures should be taken to prevent it from splashing. The evaporation flask should be filled to no more than ½ of its volume, and the bath should be cold in the beginning of evaporation. Without tert-butanol, no homogeneous solution can be obtained.

The point of co-evaporations was to remove epichlorohydrin (harmful to Step 2) as completely as possible. Epichlorohydrin is known to form a lower boiling azeotropic mixture with toluene. Each co-evaporation requires approximately 1 h to complete the removal of solvents from the viscous residual syrup. The indicated amounts of tetrahydrofuran and tert-butanol were used together in several portions; a gentle warming can be applied to facilitate the dissolution of syrup. When diluted with toluene, the mixture remains clear. Addition of tert-butanol is very efficient for homogenization of the mixture and harmless for the following dehydrochlorination. Using more tert-butanol, it would be possible to avoid the use of THF. An energetic stirring is preferred for the reaction. The mixture becomes rather viscous in the course of dehydrochlorination, so a corresponding design of the stirrer and a powerful motor should be provided. The water used in the procedure usually contained small amounts of ice. The second extract contained only negligible amounts of TGA. It was used mostly to rinse the filter after the main phase was filtered. A fritted glass filter with the medium pore size was used. The main layer (about 850 mL) was filtered first. A Pirani 501 manometer (Edwards, UK) was used to measure vacuum between the distilling apparatus and the cold trap. This position of manometer seems to be more sensitive for determining the end of distillation.

In the first distillation, the goal was to shorten the time as much as possible, thus avoiding the decomposition of TGA. Usually, the first distillation was over in 2 h. Due to the decrease in the TGA concentration at the end of distillation, the temperature in the distilling flask increased, boosting decomposition of the polymeric residue. Volatile decomposition products decreased vacuum. The distillation was stopped when the pressure increased to 0.2 mm. About 17 g of viscous yellow residue remained in the distilling flask. TLC (silica-gel on Al-foil, CHCl₃-methanol, 92 to 8 by volume, spot detection in I₂-chamber) detected about 30% of TGA in this residue, the rest was represented by oligomeric and polymeric products.

The temperature depended significantly on the rate of vapor flow, which was normal for the distillation in high vacuum. In addition to the main fraction, about 1.5 g of lower boiling fraction with bp. less than 80° C. was collected, which contained a small impurity of a compound more mobile on TLC than triglycidylamine. Also, less than 2 g of residue remained in the distilling flask and consisted mostly of polymerized triglycidylamine. The fractionation was continued for less than 2 h.

¹H NMR (CDCl₃) indicated two diastereomers of TGA at about 3:1 ratio. Most likely, these isomers differ in configurations of chiral centers at 2-C of glycidyl groups (RRR and SSS for the minor isomer; RRS and SSR for the main isomer).

### Example 2

### Purification of Triglycidylamine

Another way to purify TGA and avoid the high-vacuum distillation is by crystallization. Crude triglycidylamine (TGA) prepared from ammonia and epichlorohydrin (as described in Example 1) contains up to 15% of oligomeric impurities less mobile than TGA on TLC (silica gel, CHCl₃—MeOH, 92:8). These impurities are also less volatile than TGA and can be easily removed by high-vacuum distillation. However, complications possible in scaling up the high-vacuum distillation prompted inventors to look for other methods suitable for purification of crude TGA on a large scale. It was noticed that TGA can be crystallized from toluene or toluene-hexane solutions at low temperatures. However such crystallization was found not able to remove all the impurities of oligomers, especially the compound with R_{f} near 0.5. Thus, a combination method was used as described below, including filtration of crude TGA dissolved in a suitable solvent through a layer of silica gel. When a correct solvent system is chosen, most of the low mobile impurities can be efficiently removed, including the most stubborn impurity mentioned above. It was observed that a mixture of CHCl₃ and hexane (1:2 by volume) gives the best results. When crude TGA is dissolved in either toluene or CHCl₃ (non-diluted), the purification is much worse, and the stubborn oligomer can be removed only partially. Further optimization would be reasonable, with the aim to decrease the volumes of solvents and the amount of silica gel. After removal of the lower mobile impurities, TGA was crystallized from a mixture toluene-hexane (ca. 2:3 by volume) at −10 to −15° C., giving the compound of essentially the same purity and almost in the same yield as in the high-vacuum distillation. The typical experiment is described below.

### Isolation of TGA from the Crude Product

All the solvents were of HPLC grade, obtained from either Sigma-Aldrich Co. (St. Louis, Mo.) (e.g., toluene) or Fisher Scientific (Hampton, N.H.) (e.g., hexane, chloroform). Narrow pore silica gel for flash chromatography was obtained from VWR (West Chester, Pa.) (Catalog # JT 7024-1). Crude TGA (ca. 130 g of solvent-free product) was obtained as previously, diluted with toluene to 186.2 g (ca. 30% of toluene by wt., to increase the stability on storage) and stored at −15° C. After several days of storage, the crude TGA became partly crystalline and required warming to ca. 45° C. (until clear) before taking the aliquots. A portion (20.74 g) of this product was diluted with chloroform (60 mL) and hexane (120 mL). A column was dry-filled with silica gel (d=2 cm, 1=12 cm, ca. 38 mL of sorbent) and pre-washed with hexane. The solution of crude TGA was filtered through the sorbent, which was then washed with a mixture of chloroform (100 mL) and hexane (200 mL). TLC of the filtrate (conditions as above) showed that the most of low mobile impurities (including the stubborn one) were removed. Additional washing the silica gel with chloroform (150 mL) gave after drying on a rotary evaporator (RE, bath temperature up to 30° C.) 1.15 g of contaminated product, which was dissolved in chloroform (5 mL) with hexane (10 mL) and similarly purified on a smaller amount of silica gel (d=0.9 cm, 1=11 cm, ca. 7 mL of sorbent pre-washed with hexane, washing with chloroform-hexane, 1:2 by volume, 30 mL). The filtrates were combined, filtered (to remove possible particles of silica gel) and dried on RE (bath to 35° C.). The residue (14.11 g) was dissolved in toluene (20 mL), diluted with hexane (30 mL) cooled in ice and seeded with crystalline TGA (obtained previously by vacuum distillation). More hexane can cause separation of a syrupy precipitate, especially after cooling. TGA (even in the liquid form) is sparingly soluble in hexane. To accelerate the crystallization, a gentle rubbing with a glass rod was applied. In ca. 10 min., the crystallization was mostly over. The suspension was left overnight at −10 to −15° C. The crystals were filtered off from the cold suspension, washed (in 3 portions) with a cold mixture of toluene (6 mL) and hexane (10 mL), with cold hexane (25 mL, in portions) and dried in vacuum (up to 0.1 mm Hg) at room temperature for 8 h. For this experiment, no special equipment for cold filtration was used but a fritted glass filter cooled to ca. −15° C. prior to the filtration. Obviously, proper equipment will increase the yield of TGA. Yield of pure TGA was 11.18 g (corresponding to 100.4 g calculated for the whole amount of crude TGA). Vacuum distillation usually gave a higher yield of TGA (ca. 110 g could be expected for the whole amount of crude TGA mentioned above). However, an additional crop of TGA can be isolated from the mother liquor. In the above experiment, drying the mother liquor (RE, to 35° C.) gave 1.77 g (15.9 g for the whole amount) of crystallizing residue, which consisted mostly of TGA (according to TLC). The product was completely TLC-pure. ¹H NMR (CDCl₃) displayed exactly the same picture as for the product purified by vacuum distillation. No organic solvents trapped in the crystals were found.

### Example 3

### Synthesis of N,N′-TETRAGLYCIDYL-1,3-DIAMINOPROPANE

N,N′-Tetraglycidyl-1,3-diaminopropane was prepared similarly to TGA (see Example 1) by treatment of 1,3-diaminopropane with an excess of epichlorohydrin in 2-propanol-water in the presence of catalytic amounts of triflate. Without isolation, the resulting tetrakis-chlorohydrin was subjected to the epoxy-ring closure, as shown on the scheme:

¹H NMR of N,N′-tetraglycidyl-1,3-diaminopropane indicates 2 different sets of protons in ratio of ca. 1:1, prossibly belonging to 2 different conformations of diglycidylamino groups (with the same and the opposite configurations at 2-C chiral centers). The difference is mostly noticeable for CH₂ protons of glycidyl groups (both of the oxirane ring and CH₂N).

A mixture of 1,3-diaminopropane (15.7 mL, 0.19 mol), water (10 mL) and 2-propanol (30 mL) was slowly added to a mixture of epichlorohydrin (104 mL, 1.32 mol), water (10 mL), 2-propanol (60 mL), 1,3-diaminopropane (0.25 mL, 3.0 mmol) and triflic acid (0.4 mL, 4.5 mmol) at 27 to 32° C. over a period of 6 h. The mixture was allowed to cool to room temperature and stirred additionally for 18 h. The solvents were removed in vacuum (up to 0.1 mm Hg) at 30° C., the residual syrup (102.7 g) was diluted with water (100 mL) and again dried in vacuum, to remove the unreacted epichlorohydrin. The residue was dissolved in THF (60 mL), diluted with toluene (30 mL), and a 50% aqueous solution of NaOH (160 g, 2 mol) was added at 19-21° C. (ice cooling) in 25 min. under a vigorous stirring. The mixture was stirred at the same temperature for 2 h, diluted with toluene (100 mL) and ice-cold water (160 mL). The organic layer was separated, dried at −15° C. over anhydrous K₂CO₃, and after filtration from the desiccant and removal of the solvents, gave 23.08 g of crude N,N′-tetraglycidyl-1,3-diaminopropane as a viscous yellowish syrup. The product was purified by flash-chromatography on silica gel in CHCl₃—MeOH (100:0 to 96:4). Yield of pure compound: 10.87 g (19%). ¹H NMR (CDCl₃) δ 1.69 (m, 2H), 2.38 (dd, 14,7 Hz, 2H), 2.52, m), 2.57 (dd, 14, 7 Hz, 2H) 2.6-2.8 (m, 4H), 2.77 (m, 4H), 2.91 (dd, 14, 3 Hz, 2H), 3.01 (dd, 14, 3 Hz, 2H), 3.10 (m, 4H).

### Example 4

### Crosslinking Biomolecules of Bioprosthesis with the Biodegradable Cross-Linking Moiety

Bovine pericardium (the bioprosthesis) was crosslinked with 100 mM TGA in 0.1 M HEPES buffer at pH 7.4 containing increasing concentrations from about 2 to about 100 mM of cystamine wherein samples were kept for 7 days at room temperature Sample sets were removed at designated timepoints, rinsed in sterile saline and then subjected to a Differential Scanning Calorimetry (DSC) analysis for determining thermal shrink temperatures (Ts) using DSC7 (Perkin-Elmer, Inc., Wilson, Conn.) with temperature ramping from 60C to 100C. Analyses were typically performed in replicates of three.

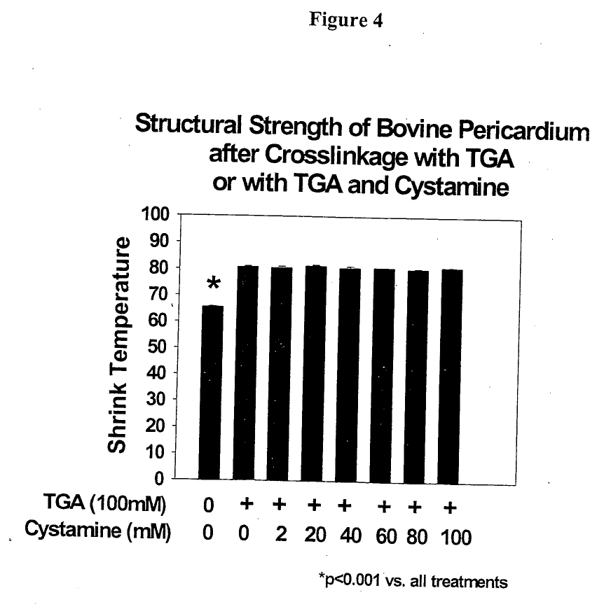

As shown in FIG. 4, TGA increased Ts significantly, and the addition of up to 100 mM of cystamine did not affect this indicator of structural strength. Therefore, the structural strength of bovine pericardium was not decreased by the addition of cystamine during cross-linking.

### Example 5

### Assessment of Biocompatibility

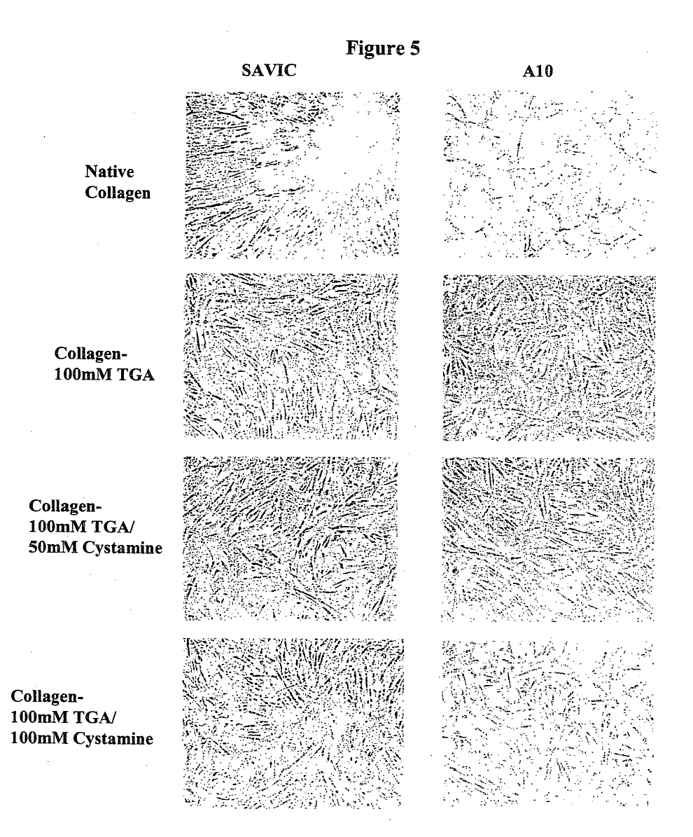

Native collagen gels were cast and subjected to either no further treatment or to TGA crosslinking in the absence of cystamine or in the presence of 50 mM and 100 mM of cystamine, followed by neutralization with 100 mM of sodium thiosulfate. Biocompatablility and biodegradation of these culture matrices were assessed in vitro using two cell lines: sheep aortic valve interstitial cells (SAVIC) and rat vascular smooth muscle cells (A10). FIG. 5 shows brightfield photomicrographs of the resulting cultures, demonstrating the excellent health of all cultures on TGA-treated matrices. Therefore, combination of TGA with cystamine does not interfere with the biocompatibility of TGA crosslinked collagen.

### Example 6

### Asssesment of Calcification and Biodegradation

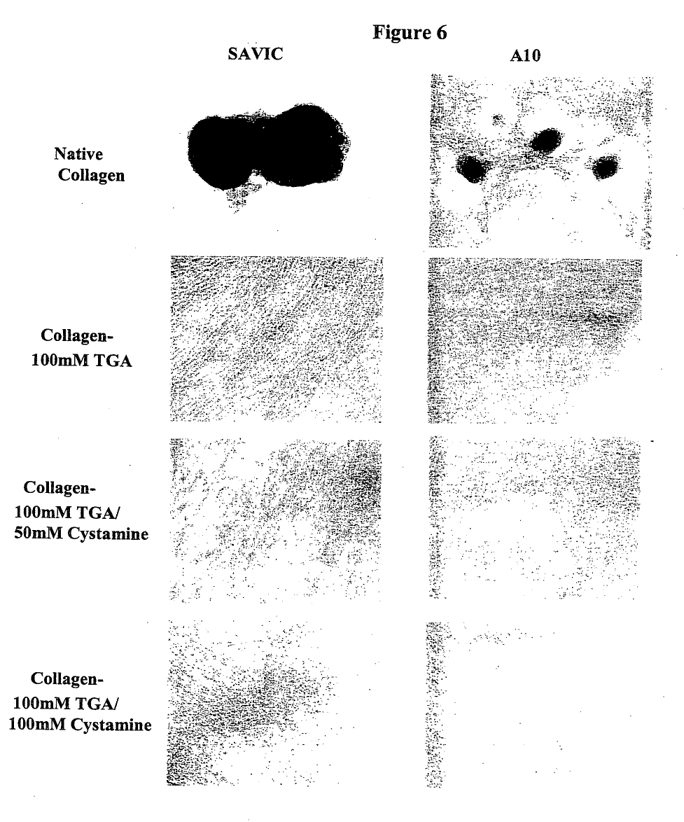

After 14 days in culture (M199 supplemented with 10% FBS), calcification was assessed by Alizarin Red staining (see FIG. 6). Gross calcification was evident in all cell cultures (SAVIC and A10) on untreated collagen, but absent when cells were grown on all TGA-cross-linked collagen either with or without cystamine addition.

Matrices remaining in the cultures after 14 days were removed completely by scraping, lyophilized and weighed. Percent weight loss for triplicate cultures was calculated vs. native collagen gels, which were incubated without being exposed to cells, and is shown in FIG. 7. Biodegradation was qualitatively enhanced by the addition of cystamine to TGA in a dose-dependent manner (A10 cells; 100 mM TGA vs. 100 mM TGA/100 mM cystamine, p=0.06) at this timepoint. All measurements were performed in triplicate. A10 cells digest the culture matrix to a lesser degree that SAVIC cells. It was observed that biodegradation, evidenced by an increase in weight loss, is enhanced by increasing of the concentration of cystamine.

1. An implantable bioprosthesis comprising biodegradably and artificially cross-linked biomolecules.

2. An implantable bioprosthesis comprising:
biomolecules having a reactive moiety and optionally a reactive group; and
a biodegradable cross-linking moiety having (a) at least two linking moieties, wherein the at least two linking moieties are non-biodegradable and (b) a spacer, wherein the spacer is biodegradable and is in communication with the at least two linking moieties, provided that the biodegradable cross-linking moiety is artificial and is covalently bound to the reactive moiety, whereby the biomolecules are cross-linked.

3. The implantable bioprosthesis of claim 2, wherein the implantable bioprosthesis is adapted to sufficiently degrade upon exposure to a cell or an enzyme at a biodegradation rate to permit an expansion of the implantable bioprosthesis and wherein the biodegradation rate is affected by an amount of the spacer.

4. The implantable bioprosthesis of claim 2, wherein at least one of the at least two linking moieties comprises an amine moiety and a hydroxyl group formed by a ring-opening reaction of an epoxide.

5. The implantable bioprosthesis of claim 2, wherein at least one of the at least two linking moieties is a member selected from the group consisting of a derivative of polyepoxy amine and a derivative of aldehyde.

6. The implantable bioprosthesis of claim 5, wherein the derivative of polyepoxy amine is a member selected from the group consisting of

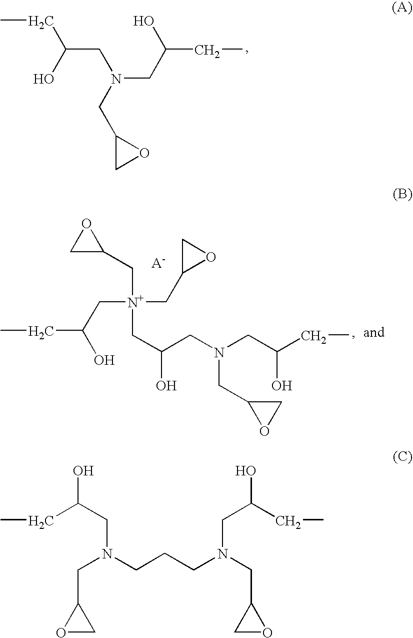

7. The implantable bioprosthesis of claim 5, wherein the derivative of polyepoxy amine is a derivative of triglycidyl amine.

8. The implantable bioprosthesis of claim 2, wherein the spacer comprises a disulfide group or a carbonyl group.

9. The implantable bioprosthesis of claim 8, wherein the spacer is represented by a formula: 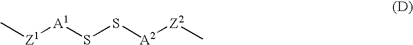
wherein A¹ and A² each comprise at least one carbon atom, and Z¹ and Z² are nucleophilic groups capable of opening an epoxy ring.

10. The implantable bioprosthesis of claim 9, wherein at least one of the nucleophilic groups is a member selected from the group consisting of an amino group, an alkylthio group, a derivative of imidazole, a derivative of pyrazole, and a derivative of pyridine.

11. The implantable bioprosthesis of claim 9, wherein the spacer is represented by a formula:
—NH—(CH₂)₂—S—S—(CH₂)₂—NH— (E)

12. The implantable bioprosthesis of claim 8, wherein the spacer is represented by a formula:

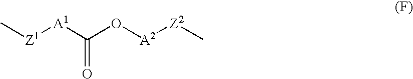

13. The implantable bioprosthesis of claim 2, wherein the biodegradable cross-linking moiety is represented by a formula:

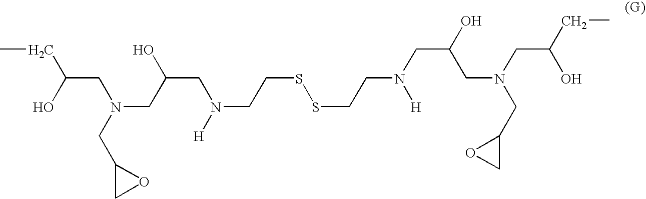

14. The implantable bioprosthesis of claim 2, wherein the reactive group is a member selected from the group consisting of an amine group, a hydroxyl group, a phosphate group, and a carboxyl group.

15. The implantable bioprosthesis of claim 2, wherein the reactive moiety is a member selected from the group consisting of an amine moiety, a hydroxyl moiety, a derivative of a phosphate moiety, and a carboxyl moiety.

16. The implantable bioprosthesis of claim 2, further comprising a non-biodegradable cross-linking moiety comprising the at least one linking moiety, provided that the non-biodegradable cross-linking moiety is free of the spacer and is covalently bound to the reactive moiety.

17. The implantable bioprosthesis of claim 16, wherein the biodegradable cross-linking moiety and the non-biodegradable cross-linking moiety are at a ratio of about 1 to about 10.

18. The implantable bioprosthesis of claim 2, wherein the implantable bioprosthesis is a member selected from the group consisting of an artificial heart, a heart valve prosthesis, an annuloplasty ring, a dermal graft, a vascular graft, a vascular stent, a structural stent, a vascular shunt, a cardiovascular shunt, a dura mater graft, a cartilage graft, a cartilage implant, a pericardium graft, a ligament prosthesis, a tendon prosthesis, a urinary bladder prosthesis, a pledget, a suture, a permanently in-dwelling percutaneous device, a surgical patch, a vascular stent, a cardiovascular stent, a structural stent, a coated stent, a vascular shunt, a cardiovascular shunt, and a coated catheter.

19. The implantable bioprosthesis of claim 18, wherein the implantable bioprosthesis is the heart valve prosthesis.

20. The implantable bioprosthesis of claim 2, wherein the biomolecules are derived from a biological tissue and or a synthetic analog of a bioprosthetic tissue.

21. The implantable bioprosthesis of claim 20, wherein the tissue is selected from the group consisting of a heart, a heart valve, an aortic root, an aortic wall, an aortic leaflet, a pericardial tissue, a connective tissue, dura mater, a bypass graft, a tendon, a ligament, a dermal tissue, a blood vessel, an umbilical tissue, a bone tissue, a fascia, and a submucosal tissue.

22. The implantable bioprosthesis of claim 21, wherein the tissue is harvested from an animal.

23. The implantable bioprosthesis of claim 22, wherein the animal is selected from the group consisting of a human, a cow, a pig, a dog, a seal, and a kangaroo.

24. The implantable bioprosthesis of claim 2, wherein the biomolecules are members selected from the group consisting of proteins, glucosoaminoglucans, and an extracellular matrix.

25. The implantable bioprosthesis of claim 2, further comprising at least one of a calcification inhibiting moiety or a glucosoaminoglycan stabilizing moiety, wherein the calcification inhibiting moiety is formed by treating the implantable bioprosthesis with a calcification inhibitor and the glucosoaminoglycan stabilizing moiety is formed by treating the implantable bioprosthesis with a glucosoaminoglycan stabilizing reagent.

26. The implantable bioprosthesis of claim 25, wherein the calcification inhibitor is a member selected from the group consisting of an Al⁺³ salt, a Fe⁺³ salt, and aminobisphosphonate.

27. The implantable bioprosthesis of claim 25, wherein the glucosoaminoglycan stabilizing reagent is a member selected from the group consisting of carbodiimide and glutaraldehyde.

28. A method of making the implantable bioprosthesis of claim 2, the method comprising:
providing an untreated bioprosthesis comprising biomolecules having reactive groups;
providing at least two molecules of a linking agent;
providing a spacing agent; and
reacting the linking agent, the spacing agent and the biomolecules, so that the biodegradable cross-linking moiety is formed between the biomolecules to provide cross-linking and thereby make the implantable bioprosthesis.

29. The method of claim 28, wherein the linking agent is represented by at least one of polyepoxy amine, aldehyde, and carbodidimide.

30. The method of claim 29, wherein the linking agent is triglycidyl amine.

31. The method of claim 28, wherein the spacing agent is represented by a formula: 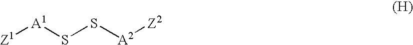
wherein A¹ and A² each comprise at least one carbon atom, and Z¹ and Z² are nucleophilic groups capable of opening an epoxy ring.

32. The method of claim 31, wherein at least one of the nucleophilic groups is a member selected from the group consisting of an amino group, an alkylthio group, a derivative of imidazole, derivative of pyrazole, and derivative of pyridine.

33. The method of claim 31 wherein the spacing agent is cystamine.

34. The method of claim 28, wherein the spacing agent is represented by a formula:

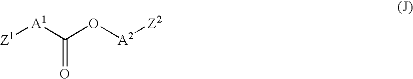

35. The method of claim 28, further comprising reacting the linking agent and the biomolecules to form the non-biodegradable cross-linking moiety, wherein the non-biodegradable cross-linking moiety cross-links biomolecules.

36. The method of claim 28, wherein the linking agent and the spacing agent are provided at a molar ratio of about 1 to about 10.

37. A method of using the implantable bioprosthesis of claim 2, the method comprising:
providing the implantable bioprosthesis;
providing an organism comprising an enzyme capable of degrading the spacer;
contacting the implantable bioprosthesis with the enzyme; and
degrading the implantable bioprosthesis and thereby permitting expansion of the implantable bioprosthesis.

38. The method of claim 37, wherein degrading of the implantable bioprosthesis proceeds at a biodegradation rate and wherein the biodegradation rate is affected by the amount of the spacer.
